(19) United States
(12) Patent Application Publication
(10) Pub. No.: US 2002/0137990 A1
(43) Pub. Date: Sep. 26, 2002

(54) Calories control apparatus

(76) Inventor: Eduardo Correa Cardoso, Rio de Janeiro (BR)

Correspondence Address: VENABLE, P.O. Box 34385, Washington, DC, 20043-9998, US

(21) Appl. No.: 09/811,648

(22) Filed: Mar. 20, 2001

(51) Int. Cl. A61B 5/00

(52) U.S. Cl. 600/300; 128/921

(57) ABSTRACT

An apparatus comprising of a scale and a computer destined to determine the existent calories in the portions of the foods, allowing the control of the daily consumption, through the weighting of the plate containing the foods to be consumed.

### BACKGROUND OF THE INVENTION

This invention relates to a machine destined to weigh and, simultaneously, to supply amount of calories of the ready foods for consumption.

The human being's need to control the amount of calories ingested daily is every time larger in the current world, governed by new beauty patterns. Besides, the obesity has been causing great evils for the humanity, increasing the incidence of diseases as infarcts, diabetes and hypertension, among others. One of the great difficulties for the combat to the obesity is related to the fact that, most of the time, the person only takes conscience that he is fat when he reaches a very big weight and this fact, by itself, is not an encourage for the change of habits and routines seeking the weigh loss. It is already consensus among the professionals of health that the individual should be stimulated for not crossing a certain limit of consumption of calories a day.

A lot of publications inform the amount of calories of the several foods, as well as most of the products have such information in their labels. The great problem is that a person consumes several products along one day, a lot of times mixed in unequal amounts, what hinders the control of the calories. The largest example of this difficulty is the self-service in the restaurants.

### SUMMARY OF THE INVENTION

To present invention it is destined to the use during the meals, weighing the plate there with the foods put, supplying the amount of calories for a certain amount of each food and the sum of the calories for all of the foods, allowing the user to prepare his meal inside of the limit of calories wanted.

It is a further object of this invention the supply of statistics of consumption of calories and of comparative data seeking to stimulate a person for the control of his weight.

### BRIEF DESCRIPTION OF THE DRAWINGS

FIG. 1 is a top plan view of the preferred embodiment of the invention in the work position.

FIG. 2 is a perspective view of the preferred embodiment of the invention completely closed.

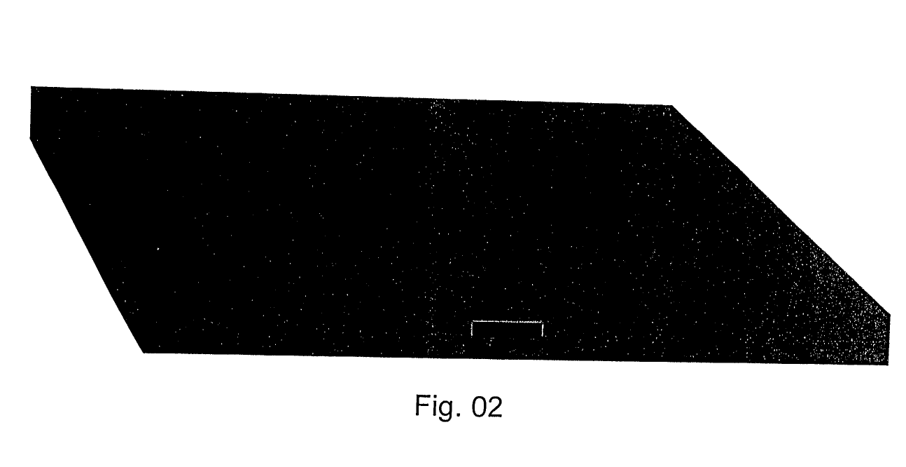

FIG. 3 is a perspective view of the preferred embodiment of the invention being opened for use.

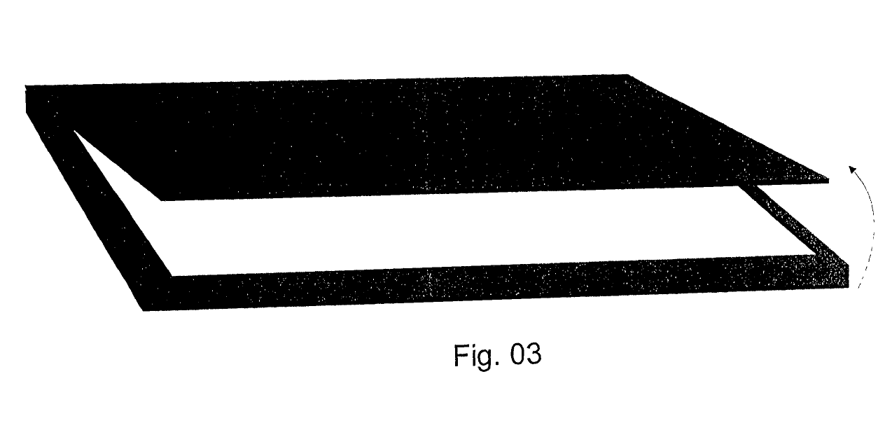

FIG. 4 is a perspective view of the second embodiment of the invention for use in restaurants.

### DETAILED DESCRIPTION

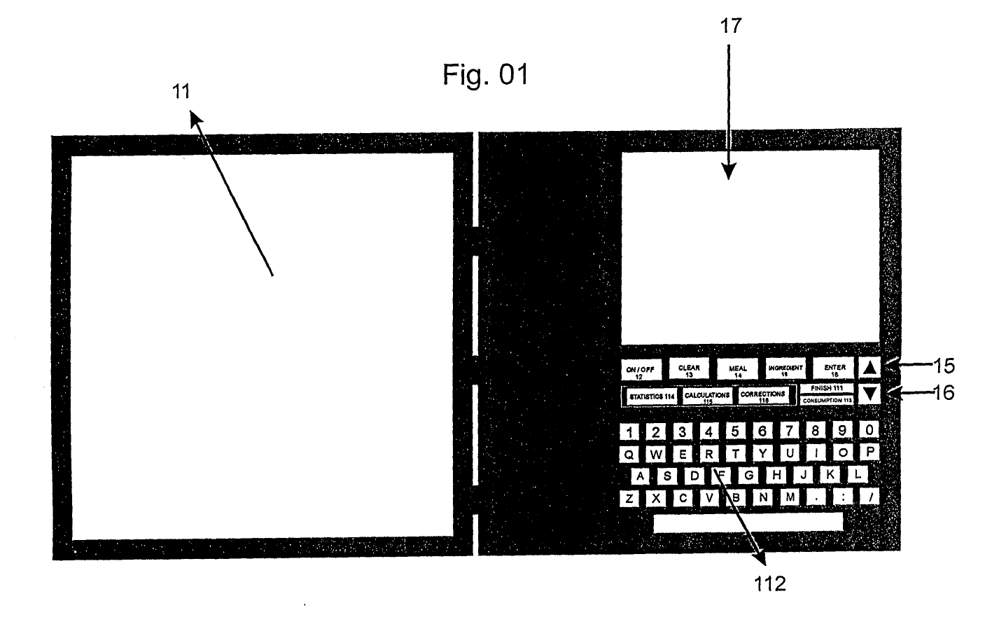

FIG. 1 illustrate the preferred embodiment of the invention in the open position, in other words, in the use position. The user presses the button “on/off” (12); then presses the button “CLEAR” (13), then puts the empty plate on the scale (11); then presses the button “meal” (14), uses the arrows (15) and (16) to choose the meal type in the viewfinder (17), confirming the choice in the key (18); then presses the button “ingredient” (19), uses the arrows (15) and (16) to choose the ingredient type in the viewfinder (17), confirming the choice in the key (18) and, finally, puts the chosen ingredient, ready to eat, on the plate. The operation is repeated for each new ingredient, pressing the button (19), using the arrows (15) and (16), visualizing in the viewfinder (17), confirming in the key (18) and putting the ingredient on the plate. To each one of these repetitions, the invention supplies in the viewfinder the amount of calories of each weight of ingredient and respective sums.

The preferred embodiment of the invention in FIG. 1 is made of synthetic material like a computer notebook. The invention works with appropriate internal electronic circuits, softwares and batteries. Their programs create database file and the user must type informations, such as name, age, sex and weight which should be typed in the keyboard (112).

When finishing composing the meal, the user presses the key “FINISH” (111), what determines the final calculation, being shown in the viewfinder (17), with the total weight of the foods, the total amount of calories, the percentile of weight and calories of each ingredient, besides the group to which the ingredient belongs. Obviously, the weight of the plate is not considered.

The key “meal” (14) makes available alternatives as breakfast, snack, lunch, dinner and desserts, while the key “ingredient” (19), makes available alternatives as vegetables, green vegetables and type of sweet. The alternatives for choices are programmed in agreement with the regional habits.

The invention is still endowed of specific keys that allow to know the daily consumption of calories (113), the statistics (114), the calculations (115) and the corrections (116).

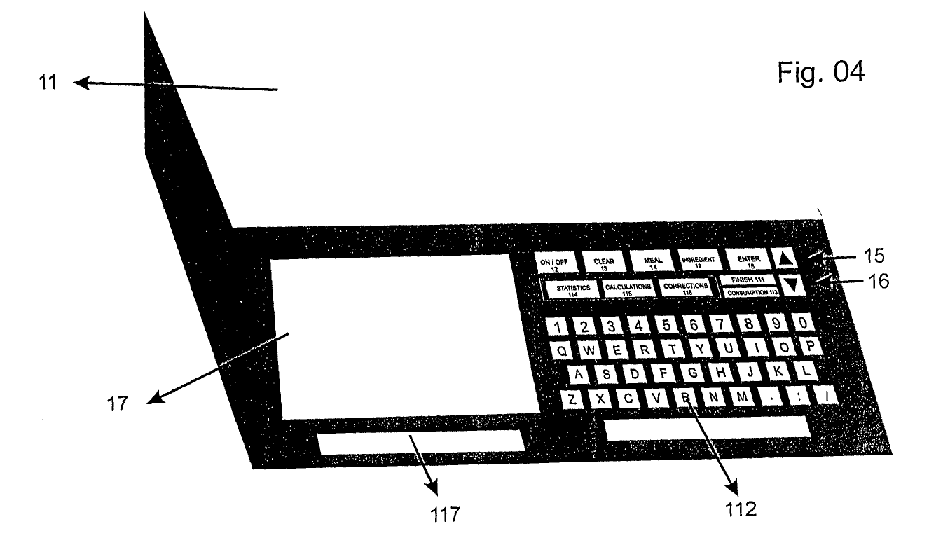

FIG. 4 illustrate an alternative embodiment of the invention for use in restaurants. This configuration is endowed with mini-printer that prints the information regarding each customer providing a ticket through de exit (117).

The invention still supplies useful pieces of advice, inducement messages in the viewfinder (17), whenever linked.

It is to be understood that while the detailed drawings and specific examples given describe preferred embodiment of the invention they are for the purpose of illustration only, that apparatus of the invention is not limited to the precise details and conditions disclosed and that various changes may be made therein without departing from the spirit of the invention as defined in the following claims.

I claim:

1. a calories control apparatus comprising:
A scale to weigh plate with food;
A keyboard for typing of data and a viewfinder;
An electronic circuit, battery and computer components for processing of the data.

2. An apparatus according to claim 1 wherein exist on a side a computer and, of other, a scale and it can be closed to the similarity of a notebook.

3. An apparatus according to claims 1 and 2 wherein the scale is placed above the keyboard and the viewfinder.

4. An apparatus according to claim 1 wherein the operation is determined by the following steps:
(a) press the key “on/off” (12);
(b) press the key “CLEAR” (13);
(c) put the empty plate on the scale (11);
(d) press the key “meal” (14);
(e) press the keys (15) and (16), visualizing in the viewfinder (17);
(f) press the key “ENTER” (18);
(g) press the key “ingredient” (19);
(h) press the keys (15) and (16), visualizing in the viewfinder (17);
(i) he press the key “ENTER” (18);
(j) put the food on the plate;
(k) repeat the steps (g) to the (j) until completing the meal and then press “FINISH” (111).

5. An apparatus according to claim 1 wherein the structure is of synthetic material while the balance and the whole components are electronic ones.
